(19) United States
(12) Patent Application Publication
(10) Pub. No.: US 2016/0166309 A1
(43) Pub. Date: Jun. 16, 2016

(54) Method and System for Determining Ablation Parameters for Ablating a Tissue

(71) Applicant: Balasubramanian K V, Bangalore (IN)

(72) Inventor: Balasubramanian K V, Bangalore (IN)

(21) Appl. No.: 14/569,395

(22) Filed: Dec. 12, 2014

(51) Int. Cl. A61B 18/12; A61B 19/00

(52) U.S. Cl. CPC A61B 18/12; A61B 19/56; A61B 2018/00577

(57) ABSTRACT

A method and system for determining ablation parameters for ablating a tissue is disclosed. In one embodiment, a method comprises determining thickness of a tissue to be ablated. The method further comprises selecting ablation temperature from a range of ablation temperature values pre-defined for the tissue to be ablated. The method comprises determining tissue temperature optimal for effective lesion formation through the entire thickness of the tissue, wherein the optimal tissue temperature corresponds to the selected ablation temperature. Moreover, the method comprises determining optimal time duration needed for the effective lesion formation through the entire thickness of the tissue at the optimal tissue temperature.

### FIELD OF TECHNOLOGY

The present disclosure generally relates to the field of ablation of tissue, and more particularly to determining ablation parameters for ablating a tissue inside an organ of a subject body.

### BACKGROUND

Ablation of tissue is a procedure used for treating a variety of clinical disorders such as cardiac arrhythmias. Typically, ablation is performed by introducing an electrode catheter having an ablative tip near a target site to be ablated. The target site is located within an organ of a patient's body (e.g., a site within patient's heart). Then, the ablative tip is brought in contact with the surface of the tissue at the target site and is actuated for a time period believed sufficient to destroy the tissue. For example, in case of treatment of cardiac arrhythmia, the ablative tip, when brought in contact with the surface of aberrant pathways, destroys the aberrant pathways which would otherwise conduct abnormal electric signals to a heart muscle.

Currently, several ablation techniques are developed which includes cryoablation, microwave ablation, radio frequency (RF) ablation, and high frequency ultrasound ablation. For example, in radio frequency (RF) ablation, the electrode catheter transfers RF energy to the tissue at the target site through the ablative tip. The RF energy generates significant heat which raises tissue temperature at the target site, resulting in ablation of the tissue at the target site. The amount of heat generated at the ablative tip during the ablation can be controlled based on the amount of RF energy transmitted through the ablative tip.

It is always desirable to form adequately-deep lesion at the target site by ablating the tissue at the target site. The adequately-deep lesion at the target site would reduce or eliminate cardiac arrhythmia typically caused due to aberrant pathways that conduct abnormal electric signals to a heart muscle. However, to form adequately-deep lesion at the target site, ablation needs to be performed at optimal tissue temperature for an optimal contact time duration.

Basically, as temperature of the ablative tip increases, contact time required to form adequately-deep lesion at the target site decreases. However, the chance of charring tissue surface and forming undesirable coagulum at the target site increases. Charring of the tissue surface may have deleterious effect on an individual undergoing this treatment. Similarly, as the temperature of the ablative tip decreases, contact time required to form an adequately-deep lesion at the target site increases. Likelihood of charring tissue surface and forming undesirable coagulum at the target site decreases. However, inadequate lesion formation may result. Inadequate lesion formation may cause leakage RF energy to pass through the layer of the tissue beneath the ablative tip. It is therefore important to ensure that the temperature of the tissue is sufficiently high for enough time to create adequately-deep lesion at the target site while preventing coagulum formation and charring of the tissue surface.

Presently, physicians monitor ablation of the tissue based on real-time ablation parameters such as ablative power, ablation temperature, and impedance. For example, temperature measurement is performed at the surface of the tissue to determine lesion formation in the tissue. In high blood flow regions, ablation temperature and tissue temperature may vary widely. Also, a steep rise in impedance value is considered as an indication of coagulation. However, a steep rise in impedance value may also be due to lack of contact between the electrode catheter with the tissue. Hence, these instinct-based monitoring techniques are unreliable. Most of the time, the physician assumes that an effective lesion is formed in the tissue and then stops the ablation when the real-time ablation parameters exceed or likely might exceed a threshold value. In these instances, the physician is not aware of time duration required for effective lesion formation.

In light of the foregoing, there exists a need for performing ablation of the tissue at optimal tissue temperature for an optimal contact time duration to form adequately-deep lesion at the target site.

### SUMMARY OF THE INVENTION

A method and system for determining ablation parameters for ablating a tissue is disclosed. According to one embodiment, a method of determining ablation parameters for ablating a tissue includes determining thickness of the tissue to be ablated. Further, the method includes selecting an ablation temperature from a range of ablation temperature values. Also, the method includes determining tissue temperature optimal for effective lesion formation through the entire thickness of the tissue, where the optimal tissue temperature corresponds to the selected ablation temperature. Moreover, the method includes determining optimal time duration needed for the effective lesion formation through the entire thickness of the tissue at the optimal tissue temperature.

Additionally, the method may include determining an amount of ablative power to be delivered to an ablative tip of an electrode catheter based on the selected ablation temperature. Also, the method may include generating a first signal indicating the amount of ablative power to be delivered to the ablative tip of the electrode catheter in contact with the tissue being ablated for the optimal time duration.

The method may further include measuring a real-time time duration during which the ablative energy is applied by the ablative tip to the tissue. The method may further include determining whether the real-time time duration exceeds the optimal time duration, and generating a second signal to discontinue supply of the ablative power to the ablative tip if the real-time time duration exceeds the optimal time duration.

The method may also include applying the ablative energy to the tissue for the optimal time duration based on the amount of the ablative power delivered at the ablative tip, resulting in effective lesion formation in the tissue.

Therein, in determining the tissue temperature optimal for effective lesion formation, the method includes the steps of:

(a) calculating a tissue temperature associated with a first layer of the tissue;

(b) determining whether the tissue temperature is less than the ablation temperature;

(c) if the tissue temperature is less than the tissue temperature, incrementing time duration required for ablating the tissue and repeating steps (a) and (b);

(d) if the tissue temperature is greater than or equal to the tissue temperature, determining whether the tissue temperature is less than a coagulation temperature;

(e) if the tissue temperature is equal to or greater than the coagulation temperature, declaring the tissue temperature as non-optimal for effective lesion formation;

(f) if the tissue temperature is less than the coagulation temperature, incrementing depth value which corresponds to a next layer in the tissue;

(g) determining whether the depth value exceeds the thickness of the tissue;

(h) if the depth value does not exceed the thickness of the tissue, repeating the steps (a) and (b); and

(i) if the depth value exceeds the thickness of the tissue, declaring the tissue temperature as optimal for the effective lesion formation.

Therein, in determining the optimal time duration for the effective lesion formation, the method may include determining the time duration taken for ablating the entire tissue at the optimal tissue temperature.

Therein, in declaring the tissue temperature as non-optimal for effective lesion formation, the method may include selecting another ablation temperature from the range of ablation temperature values, and repeating the steps (a) and (b).

Moreover, the method may include displaying the ablation temperature and the optimal time duration corresponding to the optimal tissue temperature on a display unit.

According to another embodiment, a system includes a processor, and a memory coupled to the processor, wherein the memory includes a tissue temperature determination module and a time duration determination module. The tissue temperature determination module is configured for determining thickness of tissue to be ablated, and selecting an ablation temperature from a range of ablation temperature values. Also, the tissue temperature determination module is configured for determining tissue temperature optimal for effective lesion formation through the entire thickness of the tissue, where the optimal tissue temperature corresponds to the selected ablation temperature. The time duration determination module is configured for determining optimal time duration needed for effective lesion formation through the entire thickness of the tissue at the optimal tissue temperature.

The memory may also include a power control module which is configured for determining amount of ablative power to be delivered to an ablative tip of an electrode catheter based on the selected ablation temperature, and generating a first signal indicating amount of ablative power to be delivered to the ablative tip of the electrode catheter in contact with the tissue being ablated for the optimal time duration.

The power control module further configured for measuring a real-time time duration during which ablative energy is applied by the ablative tip to the tissue. Also, the power control module configured for determining whether the real-time time duration exceeds the optimal time duration. Furthermore, the power control module configured for generating a second signal to discontinue supply (i.e. delivery) of the ablative power to the ablative tip if the real-time time duration exceeds the optimal time duration.

Therein, in determining the tissue temperature optimal for effective lesion formation, the tissue temperature determination module is configured for:

(a) calculating tissue temperature associated with a first layer of the tissue;

(b) determining whether the tissue temperature is less than the ablation temperature;

(c) if the tissue temperature is less than the tissue temperature, incrementing a time duration required for ablating the tissue and repeating steps (a) and (b);

(d) if the tissue temperature is greater than or equal to the tissue temperature, determining whether the tissue temperature is less than a coagulation temperature;

(e) if the tissue temperature is equal to or greater than the coagulation temperature, declaring the tissue temperature as non-optimal for effective lesion formation;

(f) if the tissue temperature is less than the coagulation temperature, incrementing depth value which corresponds to a next layer in the tissue;

(g) determining whether the depth value exceeds the thickness of the tissue;

(h) if the depth value does not exceed the thickness of the tissue, repeating the steps (a) and (b); and

(i) if the depth value exceeds the thickness of the tissue, declaring the tissue temperature as optimal for the effective lesion formation.

Therein, in determining the optimal time duration for the effective lesion formation, the time duration determination module is further configured for determining the time duration taken for ablating the entire tissue at the optimal tissue temperature.

Therein, in declaring the tissue temperature as non-optimal for effective lesion formation, the tissue temperature determination module is further configured for selecting another ablation temperature from the range of ablation temperature values, and repeating the steps (a) and (b).

The system further includes a display unit for displaying the ablation temperature and the optimal time duration corresponding to the optimal tissue temperature.

In yet another embodiment, a non-transitory computer-readable storage medium having machine-readable instructions stored therein, that when executed by a processor, cause the processor to perform method steps described above.

This summary is provided to introduce a selection of concepts in a simplified form that are further described below in the following detailed description. It is not intended to identify features or essential features of the claimed subject matter, nor is it intended that it be used to limit the scope of the claimed subject matter. Furthermore, the claimed subject matter is not limited to implementations that solve any or all disadvantages noted in any part of this disclosure.

### BRIEF DESCRIPTION OF THE VIEWS OF THE DRAWINGS

A more complete appreciation of the present disclosure and many of the attendant aspects thereof will be readily obtained as the same becomes better understood by reference to the following detailed description when considered in connection with the accompanying drawings:

FIG. 1 illustrates a block diagram of a catheter system for determining ablation parameters for ablating a tissue;

FIG. 2 illustrates an exemplary method of ablating a tissue in a patient's heart based on optimal tissue temperature and optimal time duration;

FIG. 3 illustrates an exemplary method of determining ablation parameters such as optimal tissue temperature and optimal time duration required for ablation of tissue; and

FIG. 4 illustrates an exemplary method of determining ablation parameters such as optimal tissue temperature and optimal time duration required for ablation of tissue.

### DETAILED DESCRIPTION

A method and system for determining ablation parameters for ablating a tissue is disclosed. Various embodiments are described with reference to the drawings, wherein like reference numerals are used to refer the drawings, wherein like reference numerals are used to refer to like elements throughout. In the following description, numerous specific details are set forth such as examples of specific components, devices, methods, etc., in order to provide a thorough understanding of embodiments of the present invention. It will be apparent, however, to one skilled in the art that these specific details need not be employed to practice embodiments of the present invention. In other instances, well-known materials or methods have not been described in detail in order to avoid unnecessarily obscuring embodiments of the present disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The terms ‘ablation site’ and ‘target site’ are interchangeably used in this document. Also, the terms ‘adequately deep lesion’ and ‘sufficiently deep lesion’ are interchangeably used throughout this document.

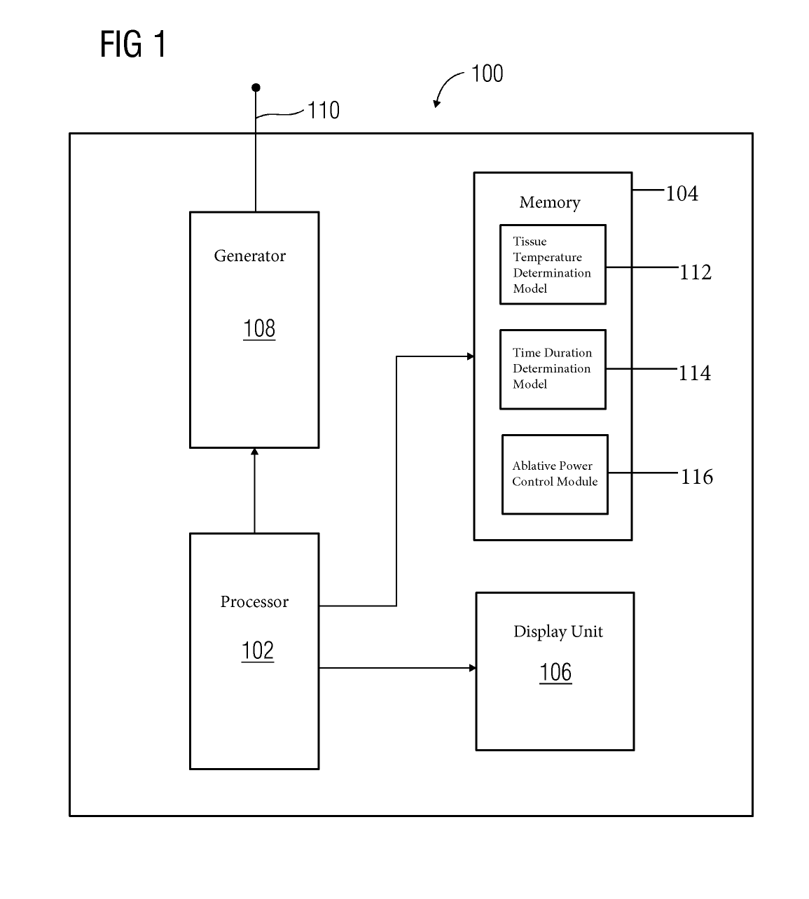

FIG. 1 is a block diagram of a catheter system 100 for determining ablation parameters for ablating a tissue, according to an embodiment. The catheter system 100 includes a processor 102, memory 104, display unit 106, generator 108, and electrode catheter 110.

It is noted that some of the components typical of conventional catheter systems may be shown in simplified form or not shown at all in FIG. 1 for purposes of brevity. Such components may nevertheless also be provided as part of, or for use with catheter system 100. Further, such components are well understood in the field of medical devices and therefore further discussion herein is not necessary for a complete understanding.

Processor 102 may comprise one or more processors, such as a single central-processing unit, or a plurality of processing units commonly referred to as a parallel processing environment. Each of the processor(s) may be any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a graphics processor, a digital signal processor, or any other type of processing circuit. The processor 102 may also include embedded controllers, such as generic or programmable logic devices or arrays, application specific integrated circuits, single-chip computers, and the like.

Memory 104 may be volatile memory and/or non-volatile memory. The memory 104 may be coupled for communication with processor 102. Processor 102 may execute instructions and/or code stored in memory 104. A variety of computer-readable storage media may be stored in and accessed from memory 104. Memory 104 may include any suitable elements for storing data and machine-readable instructions, such as read only memory, random access memory, erasable programmable read-only memory, electrically erasable programmable read only memory, a hard drive, a removable media drive for handling compact disks, digital video disks, diskettes, magnetic tape cartridges, memory cards, and the like. In the present embodiment, memory 104 comprises a tissue temperature determination module 112, a time duration determination module 114, and an ablative power control module 116. Modules 112, 114, and 116 may be stored in the form of machine-readable instructions on any of the above-mentioned storage media and may be in communication to and executed by processor 102.

Generator 108 may be a radio frequency (RF) generator connected to the electrode catheter 110. The generator 108 may be operated to emit ablative energy, such as electrical energy (e.g., RF current), near the ablative tip of the electrode catheter 110. It is noted that although the one or more of the present embodiments are described herein with reference to RF current, other types of ablative energy may also be used. The generator 108 may receive communication signal from processor 102 to emit ablative energy or not. The electrode catheter 110 is a device which is inserted into the patient, as for example, for ablating a tissue in the patient's heart.

During an exemplary ablation procedure, a user (e.g., the patient's physician or a technician) may insert the electrode catheter 110 into one of the patient's blood vessels, e.g., through the leg or the neck. The user, guided by a real-time fluoroscopy imaging device or other localization system, moves the electrode catheter 110 into an organ comprising tissue to be ablated (e.g., patient's heart). When the electrode catheter 110 reaches the patient's heart, tissue to be ablated is located. After locating the tissue, the user moves the electrode catheter 110 into contact and electrically couples the ablative tip of catheter 110 with the tissue to be ablated before applying ablative energy to form a lesion.

According to one or more of the present embodiments, the tissue temperature determination module 112 determines thickness of the tissue to be ablated. In an exemplary implementation, the tissue temperature determination module 112 may obtain the thickness of the tissue as determined by ultrasound equipment. It can be noted that other known techniques can also be used for determining the thickness of the tissue. Then, the tissue temperature determination module 112 selects an ablation temperature from a range of ablation temperature values. For example, the range of values may vary from 55° C. to 90° C. In an exemplary implementation, a minimum ablation temperature (i.e., 55° C.) is selected to determine whether corresponding tissue temperature is optimal for effective lesion formation through the entire thickness of the tissue. The “effective lesion formation” refers to when a sufficiently deep lesion is formed in the tissue without causing charring of the tissue and/or undesirable coagulum at ablation site. For example, a sufficiently deep lesion formed in a cardiac tissue, via coagulation necrosis, may result in lessening or elimination of undesirable ventricular tachycardia. The tissue temperature is considered as ‘optimal’ when a sufficiently deep lesion can be formed across the entire thickness of the tissue at such a temperature without causing charring of the tissue and/or forming undesirable coagulum.

The tissue temperature determination module 112 calculates tissue temperature across the entire thickness of the tissue. The tissue temperature determination module 112 determines whether the tissue temperature is optimal for effective lesion formation through the entire thickness of the tissue. It can be noted that, if the corresponding tissue temperature is found to be non-optimal for effective lesion formation, then the tissue temperature determination module 112 discards selected ablation temperature and selects a higher ablation temperature from the range of ablation temperature values. Accordingly, the tissue temperature determination module 112 computes tissue temperature of the tissue and determines whether the tissue temperature corresponding to the higher ablation temperature is optimal for effective lesion formation.

The time duration determination module 114 calculates the optimal time duration needed for effective lesion formation through the entire thickness of the tissue at the optimal tissue temperature. The “optimal time duration” is the time period during which ablative energy is applied to the tissue via the ablative tip to form sufficiently deep lesion in the tissue at the optimal tissue temperature. In other words, the time duration is considered as ‘optimal’ when a sufficiently deep lesion can be formed in the tissue which is the optimal tissue temperature without causing charring of the tissue and/or formation of undesirable coagulum at the ablation site. The display unit 106 may display the selected ablation temperature, the optimal tissue temperature, and/or the optimal time duration for performing ablation of the tissue to the user. As shown in FIG. 1, processor 102 may instruct, as through communication, this information to display unit 106. It can be noted that the ablation parameters such as optimal tissue temperature and the optimal time duration may be computed prior to the ablation procedure. Alternatively, the ablation parameters can be computed during the ablation procedure but prior to applying ablative energy to tissue to be ablated.

The power control module 116 computes ablative power sufficient to maintain the optimal tissue temperature across the entire thickness of the tissue. It can be noted that, the ablative power is proportional to the ablation temperature corresponding to the optimal tissue temperature. The relationship between different values of ablative power and corresponding values of ablation temperature can be maintained in a look-up table (not shown) which may be stored in memory 104. Power control module 116 may compute the value of ablative power based on the ablation temperature corresponding to the optimal tissue temperature using the look-up table.

Accordingly, power control module 116 generates a first signal indicating the ablative power to be delivered to the electrode catheter 110 which is in contact with the layer of the tissue. Processor 102 communicates the first signal to generator 108 upon which the generator 108 delivers the computed amount of ablative power (e.g., RF current) to the ablative tip of the electrode catheter 110. Consequently, the ablative tip attains the ablation temperature corresponding to the optimal tissue temperature. Thus, the ablative tip, in contact with the tissue, applies the ablative energy to the tissue for the optimal time duration such that real-time tissue temperature is substantially equal to the optimal tissue temperature. As a result, an effective lesion through the entire thickness of the tissue is formed.

The power control module 116 also measures real-time time duration during which the ablative tip applies the ablative energy to the tissue. The power control module 116 also determines whether the real-time time duration exceeds the optimal time duration. If the real-time time duration exceeds the optimal time duration, the power control module 116 generates a second signal to discontinue supply of the ablative power to the ablative tip. The processor 102 communicates the second signal to the generator 108 upon which it automatically discontinues supply of the ablative power to the ablative tip. As a result, no ablative energy is applied to the tissue beyond the optimal time duration, resulting in effective lesion formation.

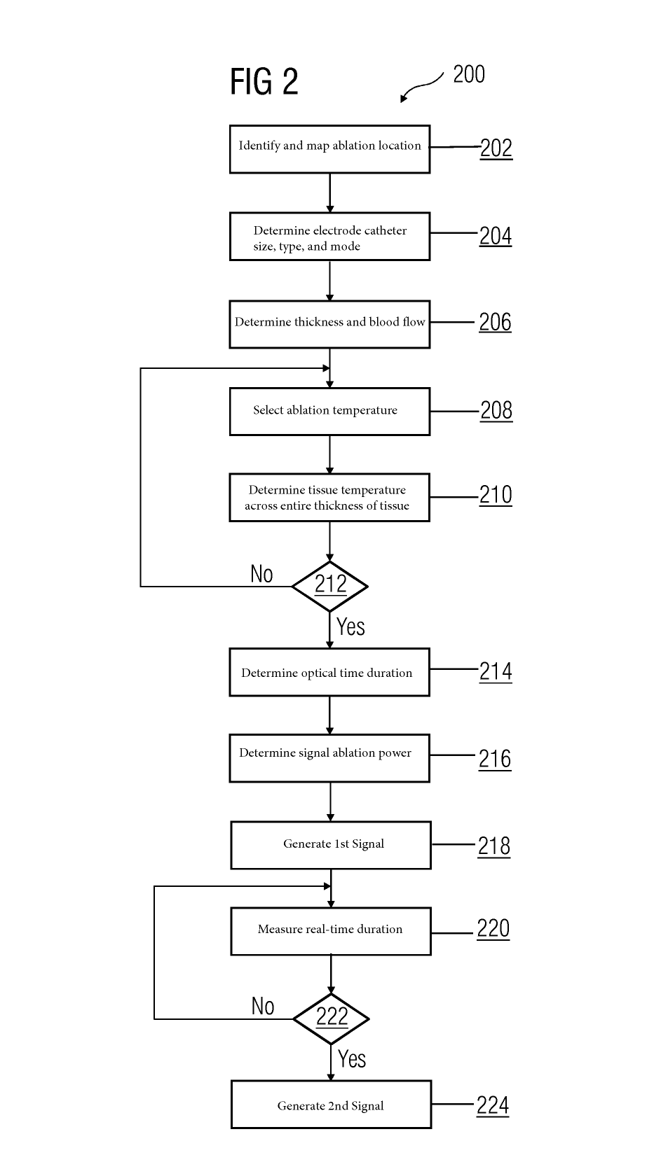

FIG. 2 is a process flowchart 200 illustrating an exemplary method of ablating a tissue in a patient's body based on optimal tissue temperature and optimal time duration, according to an embodiment. At step 202, location of tissue in the patient's body is identified and mapped. At step 204, size, type and mode of an electrode catheter to be used for ablation is determined.

At step 206, thickness of the tissue and blood flow rate, associated with the tissue, are determined. For example, the thickness of the tissue may be measured using ultrasound equipment. The blood flow rate associated with the tissue can be determined using Doppler ultrasound equipment.

At step 208, ablation temperature is selected from a range of ablation temperature values pre-defined for the tissue to be ablated. The ranges of temperature values may be different for each type of tissue. For example, the range of ablation temperature values pre-defined for the tissue located in the heart may be 55° C. to 90° C. At step 210, tissue temperature across the entire thickness of the tissue is determined. For example, the tissue temperature across the entire thickness of the tissue may be calculated using the following Bio-heat equation:

$$ρ c ( \frac{\partial T}{\partial t} ) = ( \frac{\partial }{\partial x} ) k ( \frac{\partial T}{\partial x} ) + q - {Q}_{p} + {Q}_{m} \qquad ( 1 )$$

where, ρ is mass density of the tissue, c is specific heat of the tissue, k is thermal conductivity of the tissue, T is temperature of the tissue, q is the heat source, Qₚ is perfusion heat loss, Qm is metabolic heat generation, and x is depth value corresponding to a layer of the tissue with respect to the first layer of the tissue wherein temperature needs to be calculated. The value of Qm is considered insignificant for ablation and may be ignored.

The value for Qp is calculated using the following equation:

Qₚ=ω_{b}·c_{b}·(T−T_{b}) (2)

where, ω_{b} is blood perfusion per unit volume, c_{b} is specific heat of blood and T_{b} is blood temperature. In general, ω_{b} has been assumed as uniform throughout the tissue. However, the value of ω_{b} may vary across the tissue.

The value of q may be calculated using the following equation:

q=J·E (3)

where, J is current density and E is electric field intensity.

At step 212, it is determined whether the tissue temperature is optimal for effective lesion formation through the thickness of the tissue. If the tissue temperature is determined as non-optimal, the selected ablation temperature is discarded and step 208 is repeated, in which a higher ablation temperature is selected from the range of ablation temperature values.

If the tissue temperature is determined as optimal, then at step 214, optimal time duration needed for effective lesion formation through the entire thickness of the tissue is determined. At step 216, ablative power sufficient to maintain the optimal tissue temperature across the entire thickness of the tissue is determined by estimation based on the ablation temperature of the ablative tip. The ablation temperature corresponds to the optimal tissue temperature computed at step 210.

At step 218, a first signal indicating the amount of ablative power to be delivered to the electrode catheter which is in contact with the tissue is generated. Accordingly, the amount of the ablative power is delivered to the electrode catheter based on this first signal. Consequently, the ablative energy corresponding to the delivered amount of the ablative power is applied to the tissue by the ablative tip. At step 220, real-time time duration during which the ablative tip applies the ablative energy to the tissue is monitored and measured. At step 222, it is determined whether the real-time time duration during which the ablative energy is applied is greater than or equal to the optimal time duration required for effective lesion formation in the tissue.

If the real-time time duration is greater than or equal to the optimal time duration, then at step 224, a second signal to discontinue delivery of the ablative power to the electrode catheter is generated. According, the delivery of the ablative power to the electrode catheter is automatically discontinued based on the second signal. If the real-time time duration is less than the optimal time duration, then the process 200 is routed back to the step 220. In this manner, effective lesion is formed through the thickness of the tissue.

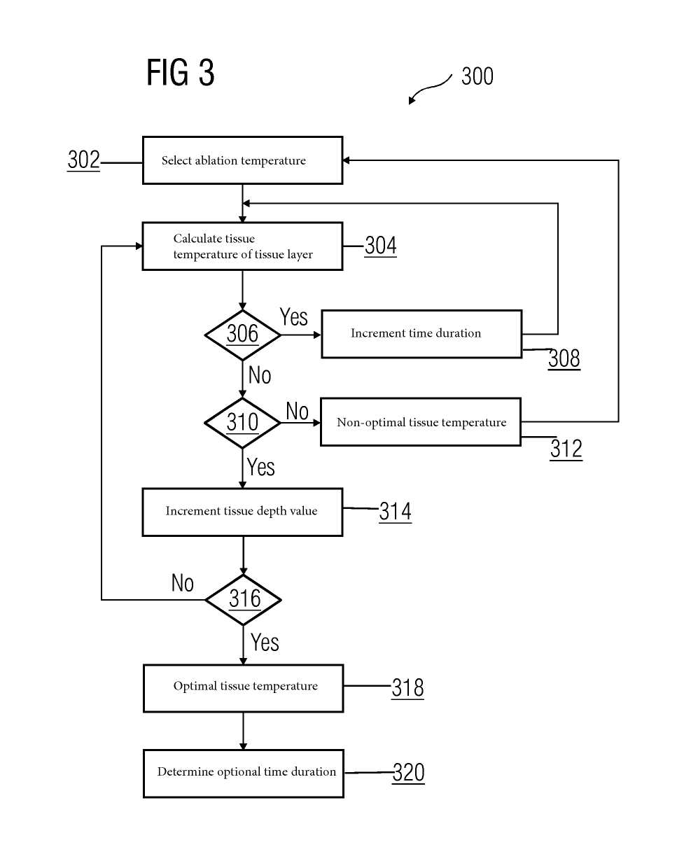

FIG. 3 is a detailed process flowchart 300 illustrating an exemplary method of determining ablation parameters such as tissue temperature and time duration required for ablation of tissue, according to an embodiment. At step 302, ablation temperature is selected from a range of ablation temperature values. For example, minimum ablation temperature from the range of ablation temperature values is selected for determining optimal tissue temperature and optimal time duration. At step 304, tissue temperature of a layer of tissue to be ablated is calculated. Consider that the layer of the tissue is a first layer and time duration for ablating the tissue as zero. Also, consider that depth value is zero as the tissue temperature is associated with the first layer of the tissue. The depth value (x) increases from first layer to last layer of the tissue.

At step 306, it is determined whether the tissue temperature associated with the layer of the tissue is less than the selected ablation temperature. If it is determined that the tissue temperature is less than the ablation temperature, then at step 308, time duration is incremented by a pre-defined value. For example, the time duration may be incremented by five seconds. From there, process 300 then repeats beginning at step 304.

If it is determined that the tissue temperature is equal to or greater than the ablation temperature, then at step 310, it is determined whether the tissue temperature is less than the coagulation temperature. The “coagulation temperature” is a temperature at which there is a chance of charring surface of the tissue and thus forming undesirable coagulum. If the tissue temperature is equal to or greater than the coagulation temperature, then at step 312, the tissue temperature is declared as non-optimal for effective lesion formation and the selected ablation temperature is discarded. Upon performing step 312, the process 300 is routed to step 302 wherein a higher ablation temperature is selected from the range of ablation temperature values.

If the tissue temperature is less than the coagulation temperature, then at step 314, the depth value which corresponds to the next layer of the tissue is incremented. At step 316, it is determined whether the incremented depth value corresponding to the next layer of the tissue is greater than or equal to the thickness of the tissue. If the incremented depth value is greater than or equal to the thickness of the tissue, then at step 318, the tissue temperature is optimal for effective lesion formation. At step 320, the optimal time duration taken for effective lesion formation through the thickness of the tissue at the optimal tissue temperature is determined. If the incremented depth value is less than the thickness of the tissue, then steps 304 to 316 are repeated until the depth value becomes equal to the thickness of the tissue.

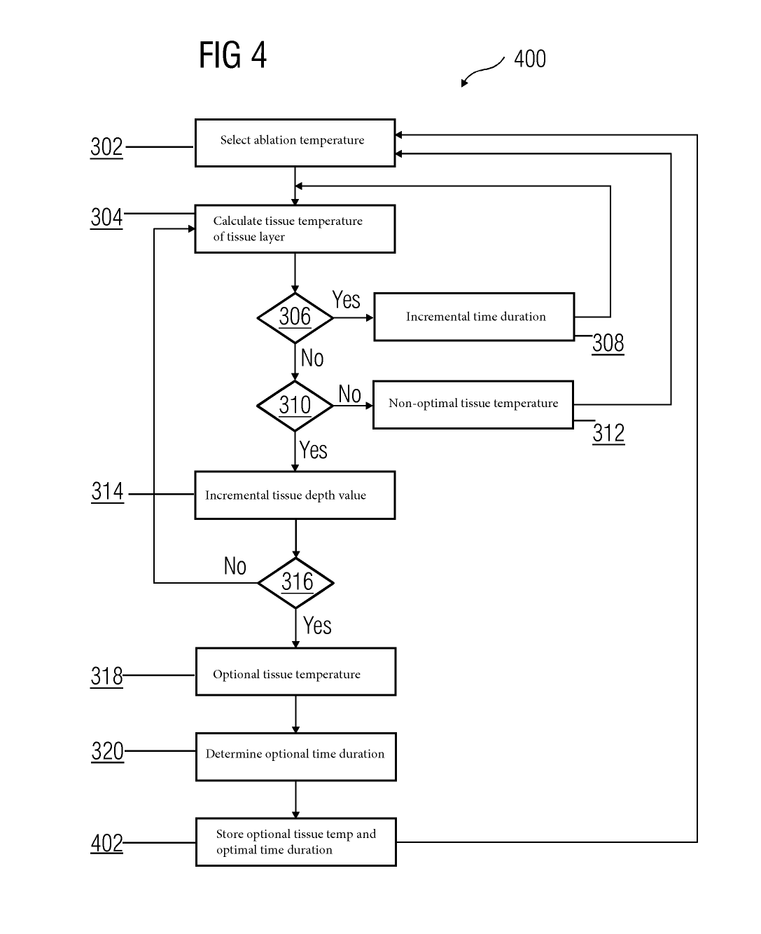

FIG. 4 is a detailed process flowchart 400 illustrating an exemplary method of determining ablation parameters such as tissue temperature and time duration required for ablation of tissue, according to another embodiment. The process 400 of FIG. 4 is similar to the process 300 of FIG. 3, except in the process 400 the optimal tissue temperature and the optimal time duration corresponding to the selected ablation temperature is stored at step 402 and the step 302 is repeated, in which a higher ablation temperature is selected from the range of the ablation temperature values. Based on the higher ablation temperature, optimal tissue temperature and optimal time duration is determined and stored. In this manner, optimal tissue temperature and optimal time duration is determined and stored for one or more of the ablation temperature values. A user (e.g., a physician) may select one among ablation temperature—time duration pairs for ablating the tissue.

It is to be understood that the system and methods described herein may be implemented in various forms of hardware, software, firmware, special purpose processors, or a combination thereof. One or more of the present embodiments may take a form of a computer program product comprising program modules accessible from computer-usable or computer-readable medium storing program code for use by or in connection with one or more computers, processors, or instruction execution system. For the purpose of this description, a computer-usable or computer-readable medium can be any apparatus that can contain, store, communicate, propagate, or transport the program for use by or in connection with the instruction execution system, apparatus, or device. The medium can be electronic, magnetic, optical, electromagnetic, infrared, or semiconductor system (or apparatus or device) or a propagation mediums in and of themselves as signal carriers are not included in the definition of physical computer-readable medium include a semiconductor or solid state memory, magnetic tape, a removable computer diskette, random access memory (RAM), a read only memory (ROM), a rigid magnetic disk and optical disk such as compact disk read-only memory (CD-ROM), compact disk read/write, and DVD. Both processors and program code for implementing each aspect of the technology can be centralized or distributed (or a combination thereof) as known to those skilled in the art.

While the present disclosure has been described in detail with reference to certain embodiments, it should be appreciated that the present disclosure is not limited to those embodiments. In view of the present disclosure, many modifications and variations would be present themselves, to those skilled in the art without departing from the scope of the various embodiments of the present disclosure, as described herein. The scope of the present disclosure is, therefore, indicated by the following claims rather than by the foregoing description. All changes, modifications, and variations coming within the meaning and range of equivalency of the claims are to be considered within their scope.

1. A method of determining ablation parameters for ablating a tissue, comprising:
determining a thickness of the tissue to be ablated;
selecting an ablation temperature from a range of ablation temperature values pre-defined for the tissue to be ablated;
determining an optimal tissue temperature for effective lesion formation through the entire thickness of the tissue, wherein the optimal tissue temperature corresponds to the selected ablation temperature; and
determining an optimal time duration needed for the effective lesion formation through the entire thickness of the tissue at the optimal tissue temperature.

2. The method of claim 1, further comprising:
determining an amount of ablative power to be delivered to an ablative tip of an electrode catheter based on the selected ablation temperature; and
generating a first signal indicating the amount of ablative power to be delivered to the ablative tip of the electrode catheter in contact with the tissue being ablated by an ablative energy for the optimal time duration.

3. The method of claim 2, further comprising:
measuring a real-time time duration during which the ablative energy is applied by the ablative tip to the tissue;
determining whether the real-time time duration exceeds the optimal time duration; and
generating a second signal to discontinue delivery of the ablative power to the ablative tip if the real-time time duration exceeds the optimal time duration.

4. The method of claim 2, further comprising:
applying the ablative energy to the tissue for the optimal time duration based on the amount of the ablative power delivered at the ablative tip, resulting in effective lesion formation in the tissue.

5. The method of claim 1, wherein determining the optimal tissue temperature for effective lesion formation comprises:
calculating a tissue temperature associated with a first layer of the tissue;
determining whether the tissue temperature is less than the ablation temperature;
incrementing a time duration required for ablating the tissue and calculating a tissue temperature associated with a first layer of the tissue and determining whether the tissue temperature is less than the ablation temperature if the tissue temperature is less than the ablation temperature;
determining whether the tissue temperature is less than a coagulation temperature if the tissue temperature is greater than or equal to the ablation temperature;
declaring the tissue temperature as non-optimal for effective lesion formation if the tissue temperature is equal to or greater than the coagulation temperature;
incrementing a depth value which corresponds to a next layer in the tissue if the tissue temperature is less than the coagulation temperature;
determining whether the depth value exceeds the thickness of the tissue;
calculating a tissue temperature associated with a first layer of the tissue and determining whether the tissue temperature is less than the ablation temperature if the depth value does not exceed the thickness of the tissue; and
declaring the tissue temperature as optimal for the effective lesion formation if the depth value exceeds the thickness of the tissue.

6. The method of claim 5, wherein determining the optimal time duration for the effective lesion formation comprises:
determining the time duration taken for ablating the entire tissue at the optimal tissue temperature.

7. The method of claim 5, wherein declaring the tissue temperature as non-optimal for effective lesion formation further comprises:
selecting another ablation temperature from the range of ablation temperature values; and
calculating a tissue temperature associated with a first layer of the tissue and determining whether the tissue temperature is less than the ablation temperature.

8. The method of claim 1, further comprising:
displaying the ablation temperature and the optimal time duration corresponding to the optimal tissue temperature on a display unit.

9. A system comprising:
a processor; and
a memory coupled to the processor, wherein the memory comprises:
a tissue temperature determination module configured for:
determining thickness of tissue to be ablated;
selecting an ablation temperature from a range of ablation temperature values pre-defined for the tissue to be ablated; and
determining anoptimal tissue temperature eptimal-for effective lesion formation through the entire thickness of the tissue, wherein the optimal tissue temperature corresponds to the selected ablation temperature;
a time duration determination module configured for determining optimal time duration needed for effective lesion formation through the entire thickness of the tissue at the optimal tissue temperature.

10. The system of claim 9, wherein the memory comprises a power control module configured for:
determining an amount of ablative power to be delivered to an ablative tip of an electrode catheter based on the selected ablation temperature; and
generating a first signal indicating amount of ablative power to be delivered to the ablative tip of the electrode catheter in contact with the tissue being ablated by an ablative energy for the optimal time duration.

11. The system of claim 10, wherein the power control module is further configured for:
measuring a real-time time duration during which the ablative energy is applied by the ablative tip to the tissue;
determining whether the real-time time duration exceeds the optimal time duration; and
generating a second signal to discontinue delivery of the ablative power to the ablative tip if the real-time time duration exceeds the optimal time duration.

12. The system of claim 9, wherein in determining the optimal tissue temperature for effective lesion formation, the tissue temperature determination module is further configured for:
calculating a tissue temperature associated with a first layer of the tissue;
determining whether the tissue temperature is less than the ablation temperature;
incrementing a time duration required for ablating the tissue and calculating a tissue temperature associated with a first layer of the tissue and determining whether the tissue temperature is less than the ablation temperature if the tissue temperature is less than the tissue temperature;
determining whether the tissue temperature is less than a coagulation temperature if the tissue temperature is greater than or equal to the tissue temperature;
declaring the tissue temperature as non-optimal for effective lesion formation if the tissue temperature is equal to or greater than the coagulation temperature;
incrementing a depth value which corresponds to a next layer in the tissue if the tissue temperature is less than the coagulation temperature;
determining whether the depth value exceeds the thickness of the tissue;
calculating a tissue temperature associated with a first layer of the tissue and determining whether the tissue temperature is less than the ablation temperature if the depth value does not exceed the thickness of the tissue; and
declaring the tissue temperature as optimal for the effective lesion formation if the depth value exceeds the thickness of the tissue.

13. The system of claim 12, wherein in determining the optimal time duration for the effective lesion formation, the time duration determination module is further configured for determining the time duration taken for ablating the entire tissue at the optimal tissue temperature.

14. The system of claim 12, wherein in declaring the tissue temperature as non-optimal for effective lesion formation, the tissue temperature determination module is further configured for:
selecting another ablation temperature from the range of ablation temperature values; and
calculating a tissue temperature associated with a first layer of the tissue and determining whether the tissue temperature is less than the ablation temperature.

15. The system of claim 9, further comprising:
a display unit for displaying the ablation temperature and the optimal time duration corresponding to the optimal tissue temperature.

16. A non-transitory computer-readable storage medium having machine-readable instructions stored therein, that when executed by a processor, cause the processor to perform method steps comprising:
determining a thickness of tissue to be ablated;
selecting an ablation temperature from a range of ablation temperature values pre-defined for the tissue to be ablated;
determining an optimal tissue temperature optimal for effective lesion formation through the entire thickness of the tissue, wherein the optimal tissue temperature corresponds to the selected ablation temperature; and
determining optimal time duration needed for effective lesion formation through the entire thickness of the tissue at the optimal tissue temperature.

17. The storage medium of claim 16, wherein the instructions cause the processor to perform the method steps comprising:
determining an amount of ablative power to be delivered to an ablative tip of an electrode catheter based on the selected ablation temperature; and
generating a first signal indicating amount of ablative power to be delivered to the ablative tip of the electrode catheter in contact with the tissue being ablated by an ablative energy for the optimal time duration.

18. The storage medium of claim 17, wherein the instructions cause the processor to perform the method steps comprising:
measuring a real-time time duration during which the ablative energy is applied by the ablative tip to the tissue;
determining whether the real-time time duration exceeds the optimal time duration; and
generating a second signal to discontinue delivery of the ablative power to the ablative tip if the real-time time duration exceeds the optimal time duration.

19. The storage medium of claim 16, wherein in determining the tissue temperature optimal for effective lesion formation, the instructions cause the processor to perform the method steps comprising:
calculating tissue temperature associated with a first layer of the tissue;
determining whether the tissue temperature is less than the ablation temperature;
incrementing time duration required for ablating the tissue and calculating a tissue temperature associated with a first layer of the tissue and determining whether the tissue temperature is less than the ablation temperature repeating if the tissue temperature is less than the tissue temperature;
determining whether the tissue temperature is less than a coagulation temperature if the tissue temperature is greater than or equal to the tissue temperature;
declaring the tissue temperature as non-optimal for effective lesion formation if the tissue temperature is equal to or greater than the coagulation temperature;
incrementing depth value which corresponds to a next layer in the tissue if the tissue temperature is less than the coagulation temperature;
determining whether the depth value exceeds the thickness of the tissue;
calculating a tissue temperature associated with a first layer of the tissue and determining whether the tissue temperature is less than the ablation temperature if the depth value does not exceed the thickness of the tissue; and
declaring the tissue temperature as optimal for the effective lesion formation if the depth value exceeds the thickness of the tissue.

20. The storage medium of claim 19, wherein in declaring the tissue temperature as non-optimal for effective lesion formation, the instructions cause the processor to perform the method steps comprising:
selecting another ablation temperature from the range of ablation temperature values; and
calculating a tissue temperature associated with a first layer of the tissue and determining whether the tissue temperature is less than the ablation temperature.
